(19) United States
(12) Patent Application Publication
(10) Pub. No.: US 2023/0383325 A1
(43) Pub. Date: Nov. 30, 2023

(54) METHOD OF SACCHARIFYING CELLULOSE

(71) Applicant: SEIKO EPSON CORPORATION, Tokyo (JP)

(72) Inventors: Yoko NAKAI, Chino (JP); Hideki TANAKA, Chino (JP); Yoshiki NAKASHIMA, Suwa (JP); Sayaka YAMASAKI, Chino (JP)

(21) Appl. No.: 18/323,450

(22) Filed: May 25, 2023

(30) Foreign Application Priority Data
May 27, 2022 (JP) 2022-086665

(51) Int. Cl. C12P 19/14; C12P 19/04; C12Q 1/34

(52) U.S. Cl. CPC C12P 19/14; C12P 19/04; C12Q 1/34

(57) ABSTRACT

A method of saccharifying cellulose according to an embodiment of the present disclosure is a method including an adding step of adding water, a raw material containing cellulose, and an enzyme to a saccharification vessel from an addition unit to obtain a mixed solution, a stirring step of stirring the mixed solution to promote a saccharification reaction, and a detecting step of detecting a concentration of the enzyme contained in a liquid of the mixed solution using a detection unit in the stirring step, in which in the detecting step, the raw material containing cellulose is additionally added to the mixed solution from the addition unit after confirmation that the concentration of the enzyme is decreased from the concentration of the enzyme at start of the stirring step and then increased.

The present application is based on, and claims priority from JP Application Serial Number 2022-086665, filed May 27, 2022, the disclosure of which is hereby incorporated by reference herein in its entirety.

### BACKGROUND

### 1. Technical Field

The present disclosure relates to a method of saccharifying cellulose.

### 2. Related Art

In recent years, with the growing awareness of climate change and problems in industrial and economic activities caused by the climate change, there is a demand for realizing carbon-neutral or carbon-free products from the viewpoint of creating a sustainable economy and society. Under such circumstances, a method of producing sugar (biomass glucose) from a cellulose-based biomass raw material using an enzyme has attracted attention, and the sugar can be used as a raw material for biomass plastic or biomass ethanol.

In the production of sugar, when raw materials are added to a saccharification vessel at once, a large torque is required to stir the solution, and the saccharification reaction efficiency is likely to decrease. Therefore, a method of adding raw materials little by little during the reaction has been examined in the related art, and for example, JP-A-2016-214244 discloses a method of additionally adding a biomass raw material to a container during saccharification.

However, the saccharification reaction efficiency is not yet sufficient.

### SUMMARY

According to an aspect of the present disclosure, there is provided a method of saccharifying cellulose, including an adding step of adding water, a raw material containing cellulose, and an enzyme to a saccharification vessel from an addition unit to obtain a mixed solution, a stirring step of stirring the mixed solution to promote a saccharification reaction, and a detecting step of detecting a concentration of the enzyme contained in a liquid of the mixed solution using a detection unit in the stirring step, in which in the detecting step, the raw material containing cellulose is additionally added to the mixed solution from the addition unit after confirmation that the concentration of the enzyme is decreased from the concentration of the enzyme at start of the stirring step and then increased.

### BRIEF DESCRIPTION OF THE DRAWINGS

FIG. 1 is a graph schematically showing a change in enzyme concentration with time in a method of saccharifying cellulose according to the present disclosure.

FIG. 2 is a graph schematically showing a determination method in a detecting step and the timing of adding a raw material in an additional adding step.

FIG. 3 is a view schematically showing an example of a saccharification vessel capable of implementing the method of saccharifying cellulose according to the present disclosure.

FIG. 4 is a flowchart showing an example of sending a command of adding a raw material based on the enzyme concentration detected by a detection unit.

FIG. 5 is a flowchart showing an example according to the present disclosure.

### DESCRIPTION OF EXEMPLARY EMBODIMENTS

Hereinafter, embodiments of the present disclosure will be described. The embodiments below will describe examples of the present disclosure. The present disclosure is not limited to the embodiments below and includes various modifications made in a range not departing from the scope of the present disclosure. Further, not all the configurations described below are necessarily essential configurations of the present disclosure.

### 1. Method of Saccharifying Cellulose

According to an embodiment of the present disclosure, a method of saccharifying cellulose is a method including an adding step of adding water, a raw material containing cellulose, and an enzyme to a saccharification vessel from an addition unit to obtain a mixed solution, a stirring step of stirring the mixed solution to promote a saccharification reaction, and a detecting step of detecting a concentration of the enzyme contained in a liquid of the mixed solution using a detection unit in the stirring step, in which in the detecting step, the raw material containing cellulose is additionally added to the mixed solution from the addition unit after confirmation that the concentration of the enzyme is decreased from the concentration of the enzyme at the start of the stirring step and then increased.

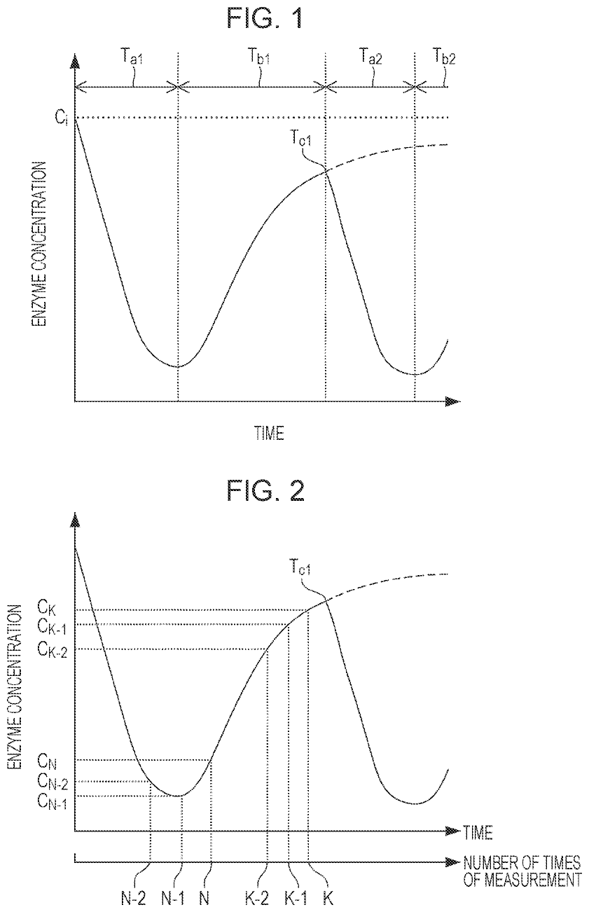

FIG. 1 is a graph schematically showing a change in enzyme concentration with time in the method of saccharifying cellulose according to the present disclosure. As shown in FIG. 1, a section Tₐ₁ where the enzyme promoting the saccharification reaction adheres to the raw material containing cellulose and the concentration of the enzyme contained in a liquid of the mixed solution is decreased is present in the saccharification reaction. Thereafter, since the raw material containing cellulose is decomposed by the saccharification reaction and the enzyme adhering to the raw material is released into the mixed solution again, the enzyme concentration is increased in a section T_{b1}. That is, the concentration of the enzyme in the mixed solution is considered to show a behavior in which the concentration is decreased once as the saccharification reaction is promoted and then increased again.

In general, when a large amount of the raw material is added to the saccharification vessel at once, a large torque is required to stir the solution, and the saccharification reaction efficiency is likely to decrease. Therefore, a method of adding raw materials little by little during the reaction has been examined in the related art. However, the timing of adding the raw material had not yet been examined in detail, and accordingly, when the raw material is added again in the section Tₐ₁ where the saccharification reaction has not been sufficiently promoted, the amount of the raw material in the saccharification vessel is likely to be excessive and thus the reaction efficiency may decrease.

On the contrary, in the present disclosure, an aspect in which the enzyme is separated in the mixed solution and the raw material is additionally added to the mixed solution after the enzyme concentration is increased (for example, the timing indicated by T_{c1} in FIG. 1) is employed. In this manner, since the raw material can be additionally added at a timing when the enzyme released into the mixed solution can be efficiently added while the initial amount of the raw material added is reduced, the reaction efficiency can be improved.

Hereinafter, each step of the method of saccharifying cellulose according to the present embodiment will be described. Further, the saccharification vessel that can be suitably used for the method of saccharifying cellulose according to the present embodiment will be described in detail.

### 1.1. Adding Step

The method of saccharifying cellulose according to the present embodiment includes an adding step of adding water, a raw material containing cellulose, and an enzyme to a saccharification vessel from an addition unit to obtain a mixed solution.

The mixed solution contains at least water, a raw material containing cellulose, and an enzyme, and other components may be added to the mixed solution so that the mixed solution contains other components.

Further, the order of adding the components is not particularly limited, but the order of initially adding the raw material, adding water, and adding the enzyme is preferable. When the components are added in such order, since the raw material and water are easily mixed and the enzyme is unlikely to be deactivated, the saccharification reaction efficiency is improved in some cases.

Further, in the adding step, stirring other than the stirring in the stirring step described below may be performed before the enzyme is added. The raw material and water are easily mixed by performing such stirring, and thus the saccharification reaction efficiency is improved in some cases.

### 1.1.1 Water

Water is not particularly limited, and examples thereof include pure water such as ion exchange water, ultrafiltration water, reverse osmosis water, or distilled water, and water obtained by reducing ionic impurities such as ultrapure water. The saccharification reaction efficiency is improved by using such water. Further, when water sterilized by irradiation with ultraviolet rays, addition of hydrogen peroxide, or the like, generation of bacteria and fungi in the mixed solution can be suppressed, and thus the saccharification reaction efficiency is improved in some cases.

The amount of water added in the adding step can be appropriately set depending on the scale and capacity of the saccharification vessel. The amount of water added is, for example, preferably 90% or less, more preferably 70% or less, and still more preferably 50% or less of the volume of the saccharification vessel. When the amount of water added is in the above-described ranges, overflow or the like of the mixed solution due to foaming of the mixed solution can be suppressed and the saccharification reaction can be stably carried out, and thus the saccharification reaction efficiency is improved in some cases.

### 1.1.2 Raw Material

The raw material contains cellulose. Further, the raw material may contain components other than cellulose, and examples thereof include components derived from wood such as lignin, hemicellulose, and processed wood components, specifically, fillers, pigments, resin components, clays, binders, toners, and oils.

Cellulose may be derived from paper, pulp, or the like. When paper is used in the raw material, the raw material can be easily prepared. Further, paper may include printed used paper. Examples of the printed used paper include copy paper, newspapers, and magazines. It is suitable to use printed used paper from the viewpoint that wastes can be reduced by saving environmental resources and reserve resources.

Paper, used paper, and the like may be added to the saccharification vessel in a pulverized state. The pulverization may be performed by, for example, cutting paper or the like with a shredder or the like or pulverizing paper or the like with a dry type defibrating machine or the like. Further, the pulverization may be performed by, for example, wet type disaggregation. When the raw material is in a pulverized or crushed state, the components other than cellulose contained in the raw material are likely to be separated from the cellulose, the cellulose is likely to come into contact with a liquid medium in the mixed solution, and thus the saccharification reaction efficiency is likely to be improved. Further, the pulverized used paper and the like are capable of improving the saccharification reaction efficiency by increasing the surface area thereof.

It is more preferable that the raw material be used in a sterilized state. Examples of a method for sterilization include a method of using high-pressure heated steam, a method of irradiation with ultraviolet rays, and a method of performing an acid treatment. In this manner, glucose and the like generated by the saccharification reaction are unlikely to be consumed by microorganisms and the like derived from the raw material, and the yield of sugar is increased in some cases.

The amount of the raw material added in the adding step is preferably in a range of 3% to 8% by mass, more preferably in a range of 3% to 7% by mass, and still more preferably in a range of 4% to 6% by mass with respect to the total amount of water added. When the amount of the raw material added in the adding step is in the above-described ranges, an increase in viscosity of the mixed solution can be suppressed, and the saccharification reaction efficiency is likely to be improved.

### 1.1.3 Enzyme

The enzyme can be used without particular limitation as long as the enzyme has a function of decomposing cellulose into sugar by cutting a β-1,4-glucoside bond. Examples of the cellulolytic enzyme include endoglucanase, cellobiohydrolase, and cellobiase (β-glucosidase). More specific examples of the cellulolytic enzyme include Cellulase SS (manufactured by Nagase ChemteX Corporation), Accellerase Duet (manufactured by GENENCOR), Cellic CTec2 (manufactured by Novozymes A/S), Cellic CTec3 (manufactured by Novozymes A/S), and Meicelase (manufactured by Meiji Seika Pharma Co., Ltd.), and a plurality of these enzymes may be used in combination. Further, xylanase may be mixed to simultaneously decompose xylan present on the surface of cellulose and increase the saccharification efficiency.

The amount of protein added to be contained in the enzyme liquid in the adding step can be appropriately set according to the amount of water or the raw material added. For example, the amount of water or the raw material added is, for example, preferably in a range of 0.01% to 0.50% by mass, more preferably in a range of 0.05% to 0.30% by mass, still more preferably in a range of 0.08% to 0.30% by mass, and particularly preferably in a range of 0.10% to 0.20% by mass with respect to the total amount of water added. According to the method of saccharifying cellulose of the present disclosure, since the enzyme can be efficiently used for the reaction, the saccharification reaction efficiency is likely to be improved even when the amount of protein added is in the above-described ranges.

### 1.1.4 Other Components

In the adding step, components other than the components described above may be added. Examples thereof include a pH adjusting agent, a surfactant, and various additive that have been typically used in the saccharification reaction.

Examples of the pH adjusting agent include one or more selected from organic acids such as acetic acid, citric acid, and phosphoric acid, inorganic acids, organic alkalis, inorganic alkalis, and salts such as sodium salts thereof, and substances constituting a buffer solution. Further, it is more preferable that the pH adjusting agent be added before the addition of the enzyme.

When the surfactant is added, it is more preferable that the surfactant include a surfactant having an antifoaming effect. The surfactant having an antifoaming effect is also referred to as an antifoaming agent. The antifoaming agent is not particularly limited, and examples thereof include a silicone-based antifoaming agent, a polysiloxane-based antifoaming agent, an acetylene glycol-based antifoaming agent, a polyether-based antifoaming agent, and a fatty acid ester-based antifoaming agent.

### 1.2 Stirring Step

The method of saccharifying cellulose according to the present embodiment includes a stirring step of stirring the mixed solution to promote the saccharification reaction.

It is preferable that the stirring in the stirring step be performed by a stirring mechanism provided in the saccharification vessel. Examples of the stirring mechanism include a magnetic stirrer, a stirrer, a stirring motor, a shaft, a stirring blade, and a combination thereof, and the stirring mechanism can be appropriately selected according to the scale and the stirring efficiency of the contents. Among these, from the viewpoint of more efficiently stirring the mixed solution and further improving the saccharification reaction efficiency, it is preferable that the stirring be performed by a stirring blade.

When the saccharification vessel includes a stirring blade, the position where the stirring blade is provided is not particularly limited. That is, the stirring blade may be provided in any of an upper portion, a side surface portion, or a bottom portion of the saccharification vessel, and a plurality of stirring blades may be provided at different positions. Among these, from the viewpoint of more efficiently stirring the immersed raw material and further improving the saccharification reaction efficiency, it is preferable that the stirring step be performed by a stirring blade provided in the bottom portion of the saccharification vessel.

Here, “stirring blade provided in the bottom portion of the saccharification vessel” denotes that the stirring blade is positioned near the lowermost portion in the space of a container of the saccharification vessel. For example, this indicates that when the distance from the lowermost portion in the space of the container of the saccharification vessel to the uppermost portion in the space of the container of the saccharification vessel is set to 1, the distance between the lowermost portion of the stirring blade and the lowermost portion in the space of the container of the saccharification vessel is preferably 0.3 or less, more preferably 0.2 or less, and still more preferably 0.1 or less.

The temperature of the mixed solution in the stirring step is preferably in a range of 30° C. to 60° C., more preferably in a range of 40° C. to 57° C., still more preferably in a range of 45° C. to 54° C., and particularly preferably in a range of 47° C. to 52° C. When the temperature thereof is in the above-described ranges, the enzyme is more activated, and the reaction efficiency is further improved in some cases.

Further, the pH of the mixed solution is preferably in a range of 4.5 to 6.0, more preferably in a range of 4.7 to 5.7, and still more preferably in a range of 4.9 to 5.5. When the pH thereof is adjusted to be in the above-described ranges, the enzyme is more activated, and the saccharification reaction efficiency is likely to be improved. In addition, when the pH value in the stirring step fluctuates, the pH thereof may be set to be in the above-described suitable ranges by adding the above-described pH adjusting agent.

### 1.3 Detecting Step

The method of saccharifying cellulose according to the present embodiment includes a detecting step of detecting the concentration of the enzyme contained in a liquid of the mixed solution using the detection unit in the above-described stirring step.

The detection in the detecting step can be performed continuously or intermittently. The interval when the detection is performed intermittently is not particularly limited, but the detection is performed preferably at intervals of 1 hour or shorter and more preferably at intervals of 30 minutes or shorter, and may be performed at intervals of 15 minutes or shorter or at intervals of 10 minutes or shorter.

The introduction of the mixed solution into the detection unit is not particularly limited, and can be performed manually or automatically. From the viewpoint of improving the handleability, it is preferable that the introduction be performed automatically and more preferable that the detection unit include a line that collects the mixed solution from the saccharification vessel. When the detection unit includes such a line, since the mixed solution can be easily automatically collected from the saccharification vessel at constant time intervals, the detecting step can be more accurately performed, and more satisfactory saccharification reaction efficiency is likely to be obtained.

One end of the line is coupled to the detection unit and the other end is coupled to the inside of the saccharification vessel. The position where one end of the line coupled to the inside of the saccharification vessel is provided is not particularly limited, but it is preferable that one end thereof be positioned in the upper portion inside the saccharification vessel. Further, it is preferable that one end of the line coupled to the inside of the saccharification vessel be oriented such that a line opening portion faces downward in the gravity direction. When the aspect in which the line is provided in the above-described manner is employed, entrance of the raw material into the line can be suppressed.

The detection unit may include one or a plurality of lines. It is preferable that the detection unit include a plurality of lines from the viewpoint that the mixed solution can be collected at a plurality of locations inside the saccharification vessel and that the detection can be more accurately performed.

Further, it is preferable that the line include a filter for removing the raw material. When the line includes such a filter, the enzyme concentration detection sensitivity is further improved, and more satisfactory saccharification reaction efficiency is likely to be obtained.

The line may include one or a plurality of filters. Further, the filter may be provided at any location on the line, but it is preferable that the filter be provided in the vicinity of both ends of the line. In this case, it is preferable that a filter with rough meshes be provided at one end of the line on the side of the saccharification vessel and that a filter with fine meshes be provided at one end of the line on the side of the detection unit. Since stirring is constantly performed inside the saccharification vessel, clogging is unlikely to occur even when a filter with rough meshes is provided at one end of the line on the side of the saccharification vessel. On the other hand, it is preferable that a filter with fine meshes be provided at one end of the line on the side of the detection unit in order to reduce the influence on the detection of the enzyme concentration.

The material of the filter is not particularly limited, and examples thereof include metals, carbon, inorganic substances, resins, and composite materials thereof. More specific examples of the material include stainless steel, polyethylene, polypropylene, polyethersulfone, polytetrafluoroethylene, polyurethane, polyester, polybutadiene, styrene-butadiene rubber, silicone rubber, natural rubber, carbon fibers, activated carbon, and a plurality of composite materials thereof.

The structure of the filter is not particularly limited, and examples thereof include a mesh structure, a woven fabric structure, a non-woven fabric structure, a sponge structure, and a porous structure. When the filter has a network structure, the aperture of the mesh may be set to, for example, 0.1 mm or greater and 10 mm or less, preferably 0.5 mm or greater and 5 mm or less, and more preferably 1 mm or greater and 3 mm or less.

The enzyme concentration can be detected by a known method, and examples thereof include an absorption spectrophotometric method, a fluorophotometric method, and a method of using gel electrophoresis.

Examples of the absorption spectrophotometric method include an ultraviolet absorption spectrophotometric method which is a method of using absorption of ultraviolet light by protein, a Bradford method which is a method of using a chemical bond between protein and a coloring dye, a WST method, a Biuret method which is a method of using a chelate complex of a copper ion generated in the presence of protein, a Lowry method, a BCA method, and a colorimetric method of using a Pierce 660 nm Protein Assay Kit (manufactured by Thermo Fisher Scientific Inc.) which is a commercially available protein quantification kit.

Examples of the fluorophotometric method include a Fluorescamine method which is a method of using a reagent emitting fluorescence by being bound to a primary amine in protein, an o-Phthalaldehyde method, a 3-(4-carboxybenzoyl)quinoline-2-carboxaldehyde (CBQCA) method, and a NanoOrange method which is a method of using a reagent emitting fluorescence by being bound to a surfactant that is applied to protein.

Examples of the method of using gel electrophoresis include a polyacrylamide gel electrophoresis method which is performed by staining protein with a dye such as Coomassie Brilliant Blue R-250 or a fluorescent dye.

Among the methods, it is preferable that the concentration of the enzyme be detected by the detection unit using the Bradford method. The Bradford method is a protein quantification method performed by using Coomassie Brilliant Blue G-250 (CBB G-250) which is a triphenylmethane-based dye. When the enzyme binds to CBB G-250, the maximum absorption wavelength of CBB G-250 is shifted from 465 nm to 595 nm, and thus the enzyme concentration can be measured with high sensitivity by measuring a change in absorbance at 595 nm. Further, the measurement operation of the Bradford method is extremely simple so that the measurement can be performed only by mixing the collected mixed solution with a CBB G-250 solution and allowing the mixed solution to stand at room temperature for 1 minute. That is, since the measurement operation is simple when the concentration of the enzyme is detected by the detection unit using the Bradford method, more satisfactory saccharification reaction efficiency is likely to be obtained.

### 1.4 Additional Adding Step

The method of saccharifying cellulose according to the present embodiment includes, in the detecting step described above, additionally adding the raw material containing cellulose to the mixed solution from the addition unit after confirmation that the concentration of the enzyme is decreased from the concentration of the enzyme at the start of the stirring step and then increased (additional adding step).

In “confirmation that the concentration of the enzyme is decreased from the concentration of the enzyme at the start of the stirring step and then increased” in the detecting step described above, the determination method is not particularly limited as long as transition from the section Tₐ₁ to the section T_{b1} shown in FIG. 1 can be confirmed, but it is preferable that the confirmation be performed based on the following determination method.

That is, the concentration of the enzyme is detected at constant time intervals in the detecting step described above, and it is determined that the concentration of the enzyme is decreased and then increased when a detected value C_{N} of the concentration in the N-th measurement and detected values C_{N-1} and C_{N-2} of the concentration one time before and two times before the N-th measurement satisfy Expressions (1) and (2).

C_{N-2}>C_{N-1} (1)

C_{N-1}<C_{N} (2)

FIG. 2 is a graph schematically showing an example of an aspect in which the confirmation is performed based on the determination method. As shown in FIG. 2, it can be determined that the enzyme concentration is shifted from a decrease to an increase when the detected values C_{N-2} of the enzyme concentration in the (N−2)th measurement is greater than the detected value C_{N-1} of the enzyme concentration in the (N−1)th measurement and the detected value C_{N-1} is less than the detected value C_{N} of the concentration in the N-th measurement. According to such a determination method, transition from the section Tₐ₁ to the section T_{b1} shown in FIG. 1 can be easily and accurately determined, and thus the enzyme released into the mixed solution can be more efficiently used and the reaction efficiency is likely to be further improved.

Further, the time interval at which the enzyme concentration is detected by the determination method is not particularly limited, but the detection is performed preferably at intervals of 1 hour or shorter and more preferably at intervals of 30 minutes or shorter, and may be performed at intervals of 15 minutes or shorter or at intervals of 10 minutes or shorter.

The timing of additionally adding the raw material containing cellulose to the mixed solution in the additional adding step is not particularly limited as long as the additional addition is performed after confirmation that the concentration of the enzyme is decreased from the concentration of the enzyme at the start of the stirring step and then increased. That is, the enzyme released into the mixed solution can be efficiently used when transition from the section Tₐ₁ to the section T_{b1} shown in FIG. 1 is confirmed, and accordingly, the timing T_{c1} of additionally adding the raw material is not particularly limited.

However, from the viewpoint that the enzyme released into the mixed solution can be more efficiently used and the reaction efficiency can be further improved, it is preferable that the additional addition be performed at the following timing. That is, the concentration of the enzyme is continuously detected at constant time intervals even after Expressions (1) and (2) are satisfied in the detecting step, and the raw material containing cellulose is additionally added to the mixed solution from the addition unit when a detected value C_{K} of the concentration in the K-th measurement (K represents an integer of greater than N) and detected values C_{K-1} and C_{K-2} of the concentration one time before and two times before the K-th measurement satisfy Expression (3).

C_{K-1}−C_{K-2}>C_{K}−C_{K-1} (3)

FIG. 2 is a graph schematically showing an example of an aspect of the addition timing. As shown in FIG. 2, it is preferable that additional addition Tₑ₁ of the raw material be performed when a difference C_{K-1}−C_{K-2} between the detected value C_{K-1} of the enzyme concentration in the (K−1)th measurement and the detected value C_{K-2} of the enzyme concentration in the (K−2)th measurement is greater than a difference C_{K}−C_{K-1} between the detected value C_{K} of the enzyme concentration in the K-th measurement and the detected value C_{K-1} of the enzyme concentration in the (K−1)th measurement. When the amount of an increase in the enzyme concentration that satisfies Expression (3) is decreased, since the saccharification reaction of cellulose is terminating and the enzyme is sufficiently released into the mixed solution, the additionally added raw material can react with the enzyme more efficiently.

Further, the addition of the raw material in the additional adding step is performed preferably at a timing of detecting an enzyme concentration of 40% or greater of the enzyme concentration (for example, Cᵢ shown in FIG. 1) in the liquid of the mixed solution at the start of the stirring step, more preferably at a timing of detecting an enzyme concentration of 45% or greater of Cᵢ, still more preferably at a timing of detecting an enzyme concentration of 50% or greater of Cᵢ, even still more preferably at a timing of detecting an enzyme concentration of 55% or greater of Cᵢ, and particularly preferably at a timing of detecting an enzyme concentration of 60% or greater of Cᵢ in the detecting step described above. In such a case, since the enzyme is sufficiently released into the mixed solution, the additionally added raw material is likely to react with the enzyme more efficiently. Further, the upper limit thereof is not particularly limited, but it is preferable that the addition of the raw material be performed until an enzyme concentration of 80% or less of Cᵢ is detected and more preferable that the addition of the raw material be performed until an enzyme concentration of 70% or less of Cᵢ is detected.

The amount of the raw material added in the additional adding step is preferably in a range of 3% to 8% by mass, more preferably in a range of 3% to 7% by mass, and still more preferably in a range of 4% to 6% by mass with respect to the total amount of water added in the adding step described above. When the amount of the raw material added in the additional adding step is in the above-described ranges, an increase in viscosity of the mixed solution can be suppressed, and the saccharification reaction efficiency is likely to be improved.

Further, when the additional adding step is not performed, the enzyme concentration gradually approaches Cᵢ as indicated by the dashed line after T_{c1} shown in FIG. 1.

### 1.5 Other Steps

According to the method of saccharifying cellulose of the present embodiment, it is preferable that the detecting step be continuously performed even after the additional adding step described above and that the additional adding step be performed again. For example, as shown in FIG. 1, the enzyme adheres to the additionally added raw materials and the concentration of the enzyme contained in the liquid of the mixed solution is decreased again (section Tₐ₂) as the behavior of the raw material after additional addition T_{c1} in the additional adding step described above. Thereafter, since the additionally added raw material is decomposed by the saccharification reaction and the enzyme adhering to the raw material is released again into the mixed solution, the enzyme concentration in the section T_{b2} is increased again. In this manner, the concentration of the enzyme in the mixed solution after the additional adding step shows again the behavior of being decreased once and then being increased again as the saccharification reaction is promoted, and thus the additional adding step can be performed a plurality of times.

The second and subsequent additional adding step can be performed in the same manner as the additional adding step described above. The number of times of performing the additional adding step is not particularly limited, but the additional adding step is performed preferably until the total amount of the raw material added in the additional adding step reaches 8% to 25% by mass, more preferably until the total amount thereof reaches 10% to 20% by mass, and still more preferably until the total amount thereof reaches 12% to 17% by mass with respect to the total amount of water added in the additional adding step described above. When the total amount of the raw material added in the additional adding step is in the above-described ranges, an increase in viscosity of the mixed solution can be suppressed, and the saccharification reaction efficiency is likely to be improved.

### 1.6 Saccharification Vessel

The saccharification vessel is not particularly limited as long as the saccharification vessel includes at least the addition unit and the detection unit and the contents can be stirred. The scale of the saccharification vessel is also not limited, and the saccharification vessel may have a laboratory scale of a beaker, a flask, or the like, a pilot plant scale, or a commercial plant scale.

The saccharification vessel may include a container and a lid. The saccharification vessel may appropriately include a port for taking out a product, a mechanism for stirring the inside, a window for observing the inside, a heater for heating and cooling, a refrigerant pipe, a jacket, and other pipes. Further, the saccharification vessel may include a liquid level meter and a thermometer and may have an opening for providing these meters.

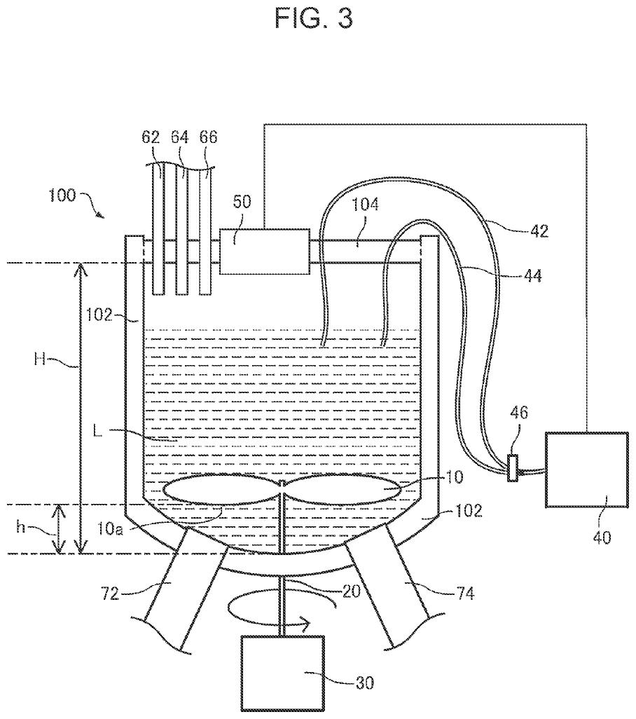

FIG. 3 is a view schematically showing an example of the saccharification vessel capable of implementing the method of saccharifying cellulose according to the present embodiment. A saccharification vessel 100 includes an addition unit formed of an addition port 50 through which the raw material is added, a tube 62 that introduces the enzyme, a tube 64 that introduces water, and a tube 66 that introduces a pH adjusting agent, and a detection unit formed of a first line 42, a second line 44, a filter 46, and a detection device 40.

### 1.6.1 Addition Unit

In the example shown in FIG. 3, the addition unit includes the addition port 50 through which the raw material is added, the tube 62 that introduces the enzyme, the tube 64 that introduces water, and the tube 66 that introduces a pH adjusting agent inside the saccharification vessel 100. The addition unit may include other constituent elements in addition to the constituent elements described above and may include, for example, a tube that introduces the surfactant. In addition, the position where the addition unit is provided is not particularly limited, but it is preferable that the addition unit be provided in the upper portion of the saccharification vessel 100.

### 1.6.2 Detection Unit

In the example shown in FIG. 3, the detection unit includes the first line 42, the second line 44, and the detection device 40. Each one end of the first line 42 and the second line 44 is coupled to the detection device 40, and the other end is coupled to the inside of the saccharification vessel 100. The first line 42 and the second line 44 includes filters for removing the raw material, and a filter 46 is provided in the vicinity of one end of the line on the side of the detection device 40 and a filter (not shown) is provided in the vicinity of one end of the line on the side of the saccharification vessel 100.

The detection device 40 measures the concentration of the enzyme in the collected liquid and is capable of sending a command to add the raw material again from the addition port 50 based on the measurement result. The measurement of the enzyme concentration in the detection device 40 can be performed by using an absorbance detector such as an ultraviolet-visible spectrophotometer or a spectrofluorometer, and the detection device 40 may include a mechanism for adding a coloring dye or a copper ion solution to the collected liquid as necessary. For example, “V-770 Series” (trade name, manufactured by Jasco Corporation) can be used as the ultraviolet-visible spectrophotometer, and for example, “F-7000” (trade name, manufactured by Hitachi High-Tech Corporation) can be used as the spectrofluorometer.

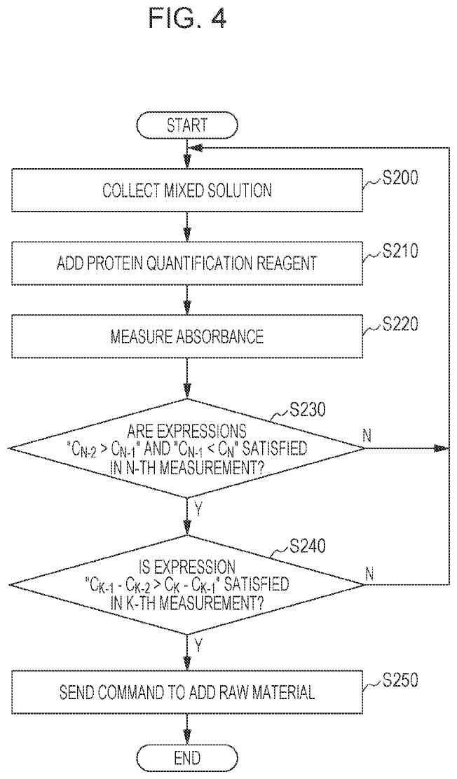

FIG. 4 is a flowchart showing an example of sending a command to add the raw material based on the enzyme concentration detected by the detection unit.

In Step S200, the mixed solution is collected at a plurality of locations inside the saccharification vessel 100 and transferred to a quartz cell in the detection device 40 by the first line 42 and the second line 44. The frequency of collecting the mixed solution is not particularly limited, but the mixed solution is collected preferably at time intervals of 1 hour or shorter, more preferably at time intervals of 30 minutes or shorter, still more preferably at time intervals of 15 minutes or shorter, and particularly preferably at time intervals of 10 minutes or shorter from the start of the stirring in the stirring step.

In Steps S210 and S220, the concentration of the enzyme contained in the liquid of the mixed solution is measured by adding a protein quantification reagent such as a coloring dye or a copper ion solution to the mixed solution transferred to the quartz cell and measuring the absorbance with an absorbance detector.

In Step S230, it is confirmed whether Expressions (1) and (2) are satisfied with reference to the enzyme concentration C_{N} detected in the N-th measurement in Step S220, the concentration C_{N-1} one time before the N-th measurement, and the concentration C_{N-2} one time before the (N−1)th measurement. When the expressions are not satisfied, the process returns to Step S200, and the mixed solution is collected again. When the expressions are satisfied, it is determined that the concentration of the enzyme in the mixed solution is decreased once and then increased, and the process proceeds to Step S240.

In Step S240, it is confirmed whether Expression (3) is satisfied with reference to the enzyme concentration C_{K} detected in the K-th measurement after the N-th measurement in Step S230, the concentration C_{K-1} one time before the K-th measurement, and the concentration C_{K-2} one time before the (K−1)th measurement. When the expression is not satisfied, the process returns to Step S200, and the mixed solution is collected again.

In Step S250, it is determined that the saccharification reaction is terminating when the relationship of Expression (3) is satisfied in the K-th measurement, and a command to add the raw material to the addition port 50 is sent so that the addition port 50 is opened and the raw material is added therethrough.

### 1.6.3 Stirring Unit

In the example shown in FIG. 3, the stirring unit includes a stirring blade 10, a stirring shaft 20, and a motor 30. In FIG. 3, a mixed solution L obtained by mixing the raw material and the like is added to the saccharification vessel 100, and the mixed solution L is stirred by rotating the stirring shaft 20 and the stirring blade 10 using the motor 30.

In the example shown in FIG. 3, the stirring blade 10 is provided such that a ratio (h/H) reaches approximately 0.17 when the distance from the lowermost portion in the space of the container in the saccharification vessel 100 to the uppermost portion in the space of the container of the saccharification vessel is defined as a distance H and the distance between a lowermost portion 10a of the stirring blade and the lowermost portion in the space of the container of the saccharification vessel is defined as a distance h.

### 1.6.4 Other Configurations

In the example shown in FIG. 3, a container 102 of the saccharification vessel 100 is closed by a lid 104, and a recovery line 72 that recovers the liquid after the reaction from the saccharification vessel 100 and a residue drain 74 that discharges residues after discharge of the liquid are provided in a lower portion of the container 102.

### 2. Example

Hereinafter, the present disclosure will be described in more detail with reference to examples, but the present disclosure is not limited thereto. Hereinafter, “%” is on a mass basis unless otherwise specified.

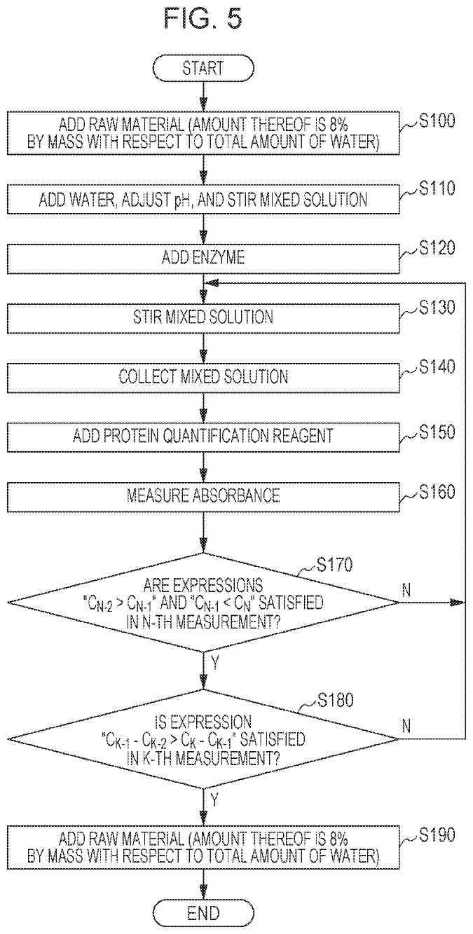

FIG. 5 is a flowchart showing an example according to the present disclosure. The example according to the present disclosure will be described with reference to FIG. 5.

In Step S100, used paper serving as the raw material containing cellulose was pulverized and added to the saccharification vessel such that the amount of the used paper reached 8% by mass with respect to the total amount of water added in Step S110 below.

In Step S110, 100 mL of water and a pH adjusting agent were added to the saccharification vessel, and the mixed solution was stirred with a magnetic stirrer at a rotation speed of 45 rpm.

In Step S120, 1.36 g of an enzyme “Cellic CTec2” (trade name, manufactured by Novozymes A/S) containing 10% by mass of protein was added to the saccharification vessel.

In Step S130, the mixed solution in the saccharification vessel was stirred so that the saccharification reaction was started. Further, the temperature of the mixed solution in the saccharification reaction was set to 49° C. and the pH thereof was set to be in a range of 5.2 to 5.5.

In Step S140, the mixed solution in the saccharification vessel was collected at time intervals of 10 minutes from the start of the stirring and transferred to a quartz cell.

In Steps S150 and S160, the concentration of the enzyme contained in the liquid of the mixed solution was measured by adding a protein quantification reagent to the mixed solution transferred to the quartz cell and measuring the absorbance with an absorbance detector.

In Step S170, it was confirmed whether Expressions (1) and (2) were satisfied with reference to the enzyme concentration C_{N} detected in the N-th measurement in Step S160, the concentration C_{N-1} one time before the N-th measurement, and the concentration C_{N-2} one time before the (N−1)th measurement. When the expressions were not satisfied, the process returned to Step S130, and the mixed solution was continuously stirred. When the expressions were satisfied, it was determined that the concentration of the enzyme in the mixed solution was decreased once and then increased, and the process proceeded to Step S180.

In Step S180, it was confirmed whether Expression (3) was satisfied with reference to the enzyme concentration C_{K} detected in the K-th measurement after the N-th measurement in Step S170 described above, the concentration C_{K-1} one time before the K-th measurement, and the concentration C_{K-2} one time before the (K−1)th measurement. When the expression was not satisfied, the process returned to Step S130, and the mixed solution was continuously stirred.

In Step S190, when the relationship of Expression (3) was satisfied in the K-th measurement, it was determined that the saccharification reaction was terminating, and used paper serving as the raw material containing cellulose was pulverized and added to the saccharification vessel such that the amount of the used paper reached 8% by mass with respect to the total amount of water added in Step S110 described above.

In the saccharification reaction in the example, the relationships of Expressions (1) and (2) were satisfied in the measurement after 30 minutes from the start of the stirring in the stirring step, and it was confirmed that the enzyme concentration was decreased and then increased. The concentration of the enzyme in the liquid of the mixed solution after 20 minutes from the start of the stirring in the stirring step was approximately 40% of the concentration of the enzyme in the liquid of the mixed solution at the start of Step S130. Further, the relationship of Expression (3) was satisfied in the measurement after 60 minutes from the start of the stirring in the stirring step, and it was confirmed that the saccharification reaction was terminating. The concentration of the enzyme in the liquid of the mixed solution after 50 minutes from the start of the stirring in the stirring step was approximately 60% of the concentration of the enzyme in the liquid of the mixed solution at the start of Step S130. Therefore, the raw material containing cellulose was additionally added after 70 minutes from the start of the stirring in the stirring step (Step S190). Thereafter, since no change in enzyme concentration with time was confirmed after 180 minutes from the start of the stirring in the stirring step, it was confirmed that the saccharification reaction was terminating, and thus the stirring was stopped. The concentration of the glucose liquid finally obtained was 65 g/L.

On the contrary, when the entire raw material was added at once and the raw material was not additionally added or when the raw material was additionally added 30 minutes before the start of the stirring in the stirring step even in a case where the raw material was additionally added, the concentration of the glucose liquid finally obtained was less than the concentration in the example described above, and the saccharification reaction efficiency was degraded.

The following contents are derived from the embodiment described above.

According to an aspect, a method of saccharifying cellulose is a method including an adding step of adding water, a raw material containing cellulose, and an enzyme to a saccharification vessel from an addition unit to obtain a mixed solution, a stirring step of stirring the mixed solution to promote a saccharification reaction, and a detecting step of detecting a concentration of the enzyme contained in a liquid of the mixed solution using a detection unit in the stirring step, in which in the detecting step, the raw material containing cellulose is additionally added to the mixed solution from the addition unit after confirmation that the concentration of the enzyme is decreased from the concentration of the enzyme at start of the stirring step and then increased.

In an aspect of the method of saccharifying cellulose described above, in the detecting step, the concentration of the enzyme may be detected at constant time intervals, and when a detected value C_{N} of the concentration in N-th measurement and detected values C_{N-1} and C_{N-2} of the concentration one time before and two times before the N-th measurement satisfy Expressions (1) and (2), it may be determined that the concentration of the enzyme is decreased and then increased.

C_{N-2}>C_{N-1} (1)

C_{N-1}<C_{N} (2)

In any one of the aspects of the method of saccharifying cellulose described above, in the detecting step, the concentration of the enzyme may be continuously detected at constant time intervals even after Expressions (1) and (2) are satisfied, and when a detected value C_{K} of the concentration in K-th measurement (K represents an integer of greater than N) and detected values C_{K-1} and C_{K-2} of the concentration one time before and two times before the K-th measurement satisfy Expression (3), the raw material containing cellulose may be additionally added to the mixed solution from the addition unit.

C_{K-1}−C_{K-2}>C_{K}−C_{K-1} (3)

In any one of the aspects of the method of saccharifying cellulose described above, the detection unit may include a line that collects the mixed solution from the saccharification vessel.

In any one of the aspects of the method of saccharifying cellulose described above, the line may include a filter that removes the raw material.

In any one of the aspects of the method of saccharifying cellulose described above, the stirring step may be performed by a stirring blade provided in a bottom portion of the saccharification vessel.

In any one of the aspects of the method of saccharifying cellulose described above, the detection unit may detect the concentration of the enzyme by a Bradford method.

The present disclosure is not limited to the above-described embodiments, and various modifications can be made. For example, the present disclosure has configurations that are substantially the same as the configurations described in the embodiments, for example, configurations with the same functions, the same methods, and the same results as described above or configurations with the same purposes and the same effects as described above. Further, the present disclosure has configurations in which parts that are not essential in the configurations described in the embodiments have been substituted. Further, the present disclosure has configurations exhibiting the same effects as the effects of the configurations described in the embodiments or configurations capable of achieving the same purposes as the purposes of the configurations described in the embodiments. Further, the present disclosure has configurations in which known techniques have been added to the configurations described in the embodiments.

What is claimed is:

1. A method of saccharifying cellulose, comprising:
an adding step of adding water, a raw material containing cellulose, and an enzyme to a saccharification vessel from an addition unit to obtain a mixed solution;
a stirring step of stirring the mixed solution to promote a saccharification reaction; and
a detecting step of detecting a concentration of the enzyme contained in a liquid of the mixed solution using a detection unit in the stirring step,
wherein in the detecting step, the raw material containing cellulose is additionally added to the mixed solution from the addition unit after confirmation that the concentration of the enzyme is decreased from the concentration of the enzyme at start of the stirring step and then increased.

2. The method of saccharifying cellulose according to claim 1,
wherein in the detecting step, the concentration of the enzyme is detected at constant time intervals, and
when a detected value C_{N} of the concentration in N-th measurement and detected values C_{N-1} and C_{N-2} of the concentration one time before and two times before the N-th measurement satisfy Expressions (1) and (2), it is determined that the concentration of the enzyme is decreased and then increased.
C_{N-2}>C_{N-1} (1)
C_{N-1}<C_{N} (2)

3. The method of saccharifying cellulose according to claim 2,
wherein in the detecting step, the concentration of the enzyme is continuously detected at constant time intervals even after Expressions (1) and (2) are satisfied, and
when a detected value C_{K} of the concentration in K-th measurement and detected values C_{K-1} and C_{K-2} of the concentration one time before and two times before the K-th measurement satisfy Expression (3), K representing an integer of greater than N, the raw material containing cellulose is additionally added to the mixed solution from the addition unit.
C_{K-1}−C_{K-2}>C_{K}−C_{K-1} (3)

4. The method of saccharifying cellulose according to claim 1,
wherein the detection unit includes a line that collects the mixed solution from the saccharification vessel.

5. The method of saccharifying cellulose according to claim 4,
Wherein the line includes a filter that removes the raw material.

6. The method of saccharifying cellulose according to claim 1,
wherein the stirring step is performed by a stirring blade provided in a bottom portion of the saccharification vessel.
